(19) United States
(12) Patent Application Publication
(10) Pub. No.: US 2004/0009528 A1
(43) Pub. Date: Jan. 15, 2004

(54) Protein chips

(76) Inventors: Shyh-Yu Shaw, Tainan (TW); Por-Hsiung Lai, Tapei (TW); Jung-Jung Ou, Tainan (TW)

Correspondence Address: FISH & RICHARDSON PC, 225 FRANKLIN ST, BOSTON, MA, 02110, US

(21) Appl. No.: 10/193,377

(22) Filed: Jul. 11, 2002

(51) Int. Cl. G01N 33/53; G01N 33/542; C12M 1/34; C07K 16/46; C07K 14/705; C07K 14/525

(52) U.S. Cl. 435/7.1; 435/287.2; 530/391.1; 530/350; 530/351

(57) ABSTRACT

This invention features a compound-bound substrate that includes a solid support having a surface; and a plurality of compounds having formula (I) covalently bound to the surface:

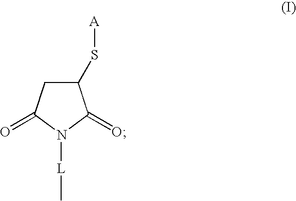

wherein L is a linking group; and A is an immunoglobulin G protein-binding molecule that is covalently bonded at one of its termini.

### BACKGROUND

The completion of the human genome project has revealed the sequences of 30,000-40,000 genes. An important task after the completion of the gene identification is to understand the function, modification, and regulation of every associated encoded protein. Currently, much effort has been focused on studying gene, and hence protein, function and regulation by analyzing mRNA expression profiles, gene disruption phenotypes, two-hybrid interactions, and protein subcellular localization. See, e.g., Ross-Macdonald et al. (1999) Nature 402: 413; DeRisi et al. (1997) Science 278: 680; Uetz et al. (2000) Nature 403: 623; and Ito et al. (2000) Proc. Natl. Acad. Sci. USA 97: 1143. Although these studies are useful, more information about protein function can be derived from the analysis of the biochemical activities of the proteins themselves by protein arrays. See, e.g., Zhu et al. (2001) Science 293: 2101; and MacBeath & Schreiber (2000) Science 289: 1760. In addition to analysis of the activities of proteins, protein arrays are useful for small molecule screening (drug discovery), drug validation, therapeutic development, and diagnostics. See, e.g., Flanagan (2002) Genetic Engineering News 22(10): 1.

### SUMMARY

This invention is based, in part, on the design and preparation of receptor protein arrays that are useful for identifying a ligand, which binds to a receptor protein, and thus modulates the receptor activity.

In one aspect, this invention features a compound-bound substrate that includes a solid support having a surface; and a plurality of compounds having formula (I) covalently bound to the surface:

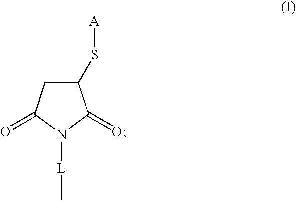

wherein L is a linking group; and A is an immunoglobulin G protein-binding molecule that is covalently bonded, e.g., at one of its termini.

In some embodiments, the compound-bound substrate comprises a chimeric protein that binds to the surface. The chimeric protein includes a polypeptide having the Fc portion of an immunoglobulin G protein, and another polypeptide having a receptor protein, such as an extracellular domain of a receptor protein (e.g., an extracellular domain of a type I membrane protein, e.g., the tumor necrosis factor-alpha protein). Such a compound-bound substrate can be used to identify a receptor binding ligand. The identification includes contacting a ligand with the compound-bound substrate and determining whether the ligand binds to the receptor protein by e.g., a fluorescence method (e.g., the ligand is fluorescence-labeled). The compound-bound substrate can also be used to identify a compound (e.g., a polypeptide or an organic molecule) that inhibits the binding of a receptor binding ligand to a receptor protein. The identification includes contacting a test compound and a receptor binding ligand with the compound-bound substrate; and determining whether the ligand binding is different from that without the presence of the test compound. Either the ligand or the test compound can be fluorescence labeled.

This invention also features a kit for testing the ability of a compound to bind to a receptor protein. The kit includes the aforementioned compound-bound substrate.

In another aspect, this invention features a substrate that includes the compound-bound substrate described above and the chimeric protein that binds to the compound-bound substrate; wherein the bound chimeric proteins have a density of at least 5×10¹⁵˜5×10¹⁶ molecules/cm².

In a further aspect, this invention features a compound-bound substrate made by a process that includes the steps of: providing a solid support having a surface that comprises a chemical group of formula -L-X; wherein L is a linker group and X is a maleimide group, i.e., —N[C(O)CH]₂; providing a plurality of immunoglobulin G protein-binding molecules, each having a mercapto group, i.e., —SH, at one of its termini; and contacting the immunoglobulin G protein-binding molecules with the surface. The process may also include the steps of: providing a chimeric protein that includes a polypeptide having the Fc portion of an immunoglobulin G protein and another polypeptide having a receptor protein; and contacting the chimeric protein with the surface.

In a further aspect, this invention features a method for preparing a substrate. The method includes the steps of: providing a surface having a plurality of molecules that include a chemical group of formula -L-NH₂, wherein L is a linking group; contacting maleic anhydride with the surface; contacting a maleimide formation reagent (i.e., one or more reagents suitable for inducing maleimide formation, e.g., ZnBr₂/HMDS) with the surface; and optionally contacting a plurality of polypeptides, oligonucleotides, or organic molecules with the surface, wherein each of the polypeptides, oligonucleotides, or organic molecules includes a mercapto group (e.g., at most one mercapto group). The mercapto group may be located at one of the termini of each polypeptide (e.g., C-terminus) or each oligonucleotide (e.g., 3′- or 5′-terminus).

Also within the scope of this invention is an array. The array includes a substrate having a plurality of addressable sites; each addressable site having a compound of formula (I) described above; each addressable site having a chimeric protein that includes a polypeptide having the Fc portion of an immunoglobulin G protein, and another polypeptide having a receptor protein, in which the chimeric protein binds to the immunoglobulin G protein-binding molecule. In some embodiments, the receptor protein is unique among each addressable site. In other embodiments, the receptor protein is identical among each addressable site.

As used herein, the term “substrate” includes both flexible and rigid solid substrates. By “flexible” is meant that the solid substrate is pliable. For example, a flexible substrate can be bent, folded, or similarly manipulated to at least some extent without breakage. The surface of a substrate can be a planar surface (e.g., a slide or a plate), a convex surface (e.g., a bead), or a concave surface (e.g., a well). Potentially useful substrates include mass spectroscopy plates (e.g., for MALDI), glass (e.g., functionalized glass, a glass slide, porous silicate glass, a single crystal silicon, quartz, or UV-transparent quartz glass), plastics and polymers (e.g., polystyrene, polypropylene, polyvinylidene difluoride, polytetrafluoroethylene, polycarbonate, PDMS, or acrylic), metal coated substrates (e.g., gold), silicon substrates, latex, membranes (e.g., nitrocellulose or nylon), and refractive surfaces suitable for surface plasmon resonance. Solid substrates can also be porous, e.g., a gel or matrix. Potentially useful porous substrates include agarose gels, acrylamide gels, sintered glass, dextran, and meshed polymers (e.g., macroporous crosslinked dextran, sephacryl, and sepharose).

The term “linking group” refers to a hydrocarbon chain or a bond. The hydrocarbon chain can be a linear or a branched alkyl chain with or without heteroatoms (e.g. N, S, or O). The hydrocarbon chain can also be an aryl chain. Examples of alkyl chains include, but are not limited to, a chain of methylene units, i.e., —(CH₂)ₙ— and n=1-12, and a chain of ethyleneglycol units, i.e., —(OCH₂CH₂)ₙ— and n=1-12. An example of an aryl chain is an annular structure of phenyl moieties, i.e.,

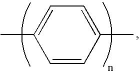

and n=1-12.

The term “immunoglobulin G protein” refers to a polypeptide that contains (1) an Fab region (including the VH, VL, and CH, domains), (2) a hinge region, and (3) an Fc portion (including CH₂ and CH₃ domains). See, e.g., U.S. Pat. No. 6,225,448. The Fc portion is the constant region on an immunoglobulin polypeptide, is located on the immunoglobulin heavy chains, and is not involved in binding to antigens, but is involved in binding to an Fc receptor.

The term “immunoglobulin G protein-binding molecule” refers to a molecule (e.g., a peptide or an organic compound) that has a high affinity (e.g., Ka of 1.0×10⁻⁷˜4.4×10⁻⁸ M) for an immunoglobulin G protein (as defined by the binding assay described in Akerstrom & Bjorck (1986) J. Biol. Chem. 261: 10240-10274). More specifically, it has a high affinity for the Fc portion of an immunoglobulin G protein. Examples of the immunoglobulin G protein-binding molecule include protein A, protein CA mouse and human high affinity immunoglobulin G receptors, an immunoglobulin G-binding domain of protein A, an immunoglobulin G-binding domain of protein G. e.g., a peptide comprising the amino acid sequence of SEQ ID NO: 1, 2, 3, 4, or 5 (See, e.g., Colbert D. et al. (1984) J. Biol. Response Modifiers 3: 255; and Olsson, A. et al. (1987) E. J Biochem 168: 319); an immunoglobulin G-binding receptors comprising the amino acid sequence of SEQ ID NO: 6 or SEQ ID NO: 7 (See, e.g., Sears D. W. et al. (1990) J. Immunol. 144(1): 371; and Peltz, G. A. et al. (1989) Proc. Natl. Acad. Sci. USA 86 (3): 1013).

| | |
|---|---|
| ADFNKQQAFYEILPNLGERNGFIQSLKDDPSLEAKKLNQAPK, | (SEQ ID NO: 1) |
| AQHDEAQQNAFYQVLNMPNLNADQRNGFIQSLKDDPSQANVLGEAEKLNDSQAPK, | (SEQ ID NO: 2) |
| TYKLILNGKTLKGETTTEAVDAATAEKVFKQYANDNGVDGEWTYDDATKTFTVTE, | (SEQ ID NO: 3) |
| TYKLVINGKTLKGETTTEAVDAATAEKVFKQYANDNGVDGEWTYDDATKTFTVTE, | (SEQ ID NO: 4) |
| TYKLVINGKTLKGETTTKAVDAETAEKAFKQYANDNGVDGVWTYDDATKTFTVTE, | (SEQ ID NO: 5) |
| MWQLLLPTALLLLVSAGMRTEDLPKAVVFLEPQWYRVLEKDSVTLKCQGA, | (SEQ ID NO: 6) |
| YSPEDNSTQWFHNENLISSQ ASSYFIDAAT VDDSGEYRCQ TNLSTLSDPV | |
| QLEVHVGWLL LQAPRWVFKEEDPIHLRCHS WRNTALHKVT YLQNGKDRKY | |
| FHHNSDFHIP KATLKDSGSY FCRGLVGSKNVSSETVNITI TQGLAVSTIS | |
| SFSPPGYQVS FCLVMVLLFA VDTGLYFSVK TNI | |
| MILTSFGDDM WLLTTLLLWV PVGGEVVNAT KAVITLQPPW VSIFQKENVT | (SEQ ID NO: 7) |
| LWCEGPHLPGDSSTQWFING TAVQISTPSY SIPEASFQDS GEYRCQIGSS | |
| MPSDPVQLQI HNDWLLLQASRRVLTEGEPL ALRCHGWKNK LVYNVVFYRN | |
| GKSFQFSSDS EVAILKTNLS HSGIYHCSGTGRHRYTSAGV SITVKELFTT | |
| PVLRASVSSP FPEGSLVTLN CETNLLLQRP GLQLHFSFYVGSKILEYRNT | |
| SSEYHIARAE REDAGFYWCE VATEDSSVLK RSPELELQVL | |
| GPQSSAPVWFHILFYLSVGI MFSLNTVLYV KIHRLQREKK YNLEVPLVSE | |
| QGKKANSFQQ VRSDGVYEEVTATASQTTPK EAPDGPRSSV GDCGPEQPEP | |
| LPPSDSTGAQ TSQS | |

An immunoglobulin G protein-binding molecule can also be a peptide containing an amino acid sequence that is at least 60% (e.g., 70%, 80%, 90%, 95%, or 98%) identical to the sequence of protein A, protein G, high affinity immunoglobulin G receptors, an immunoglobulin G-binding domain of protein A, or an immunoglobulin G-binding domain of protein G, or identical to SEQ ID NO: 1, 2, 3, 4, 5, 6, or 7 and have a high affinity for the Fc portion of an immunoglobulin G protein. The “percent identity” of two amino acid sequences can be determined using the algorithm of Karlin and Altschul (1990, Proc. Natl. Acad. Sci. USA 87: 2264-2268), modified as in Karlin and Altschul (1993, Proc. Natl. Acad. Sci. USA 90: 5873-5877). Such an algorithm is incorporated into the NBLAST and XBLAST programs (version 2.0) of Altschul et al. (1990) J. Mol. Biol. 215: 403-10. BLAST protein searches can be performed with the XBLAST program, score=50, wordlength=3 to obtain amino acid sequences homologous to the peptide molecules described herein. Where gaps exist between two sequences, Gapped BLAST can be utilized as described in Altschul et al. (1997) Nucleic Acids Res. 25(17): 3389-3402. When utilizing BLAST and Gapped BLAST programs, the default parameters of the respective programs (e.g., XBLAST and NBLAST) can be used.

A “chimeric protein” is a protein having a mixture of sequences from different sources. As used herein, the chimeric protein includes a first polypeptide containing the Fc portion of an immunoglobulin G protein, and a second polypeptide containing, e.g., a receptor protein (an extracellular domain of a receptor protein). The first polypeptide is attached at the C-terminal of the second polypeptide.

The term “receptor protein” refers to a protein, found on the surface of a cell, that interacts with a specific molecule such as a hormone, an antibody, a drug, a peptide, or a virus. A receptor protein includes at least one extracellular domain and at least one cytoplasmic domain, such as a type-I membrane protein. The “type I membrane protein” is the protein having a single transmembrane domain and its N-terminus facing the extracellular side of cells. See, e.g., Lehninger Principles of Biochemistry, 3^{rd} edition, p 401. Examples of the type I membrane proteins include tumor necrosis factor-alpha (TNF-α) receptor I, TNF-α receptor II, GM-CSF receptor, EPO receptor, EGF receptor, interleukin-4 receptor, and thrombopoietin receptor.

The invention provides one or more of the following advantages. The method for preparing a compound-bound substrate described above is economical and versatile and generates the compound-bound substrate that exhibits unexpected high homogeneity, and therefore high efficiency in binding with sequentially added chimeric proteins. Additionally, the identification a receptor binding ligand, or the identification of a compound that inhibits the binding of a receptor binding ligand to a receptor protein, can be accomplished with high efficiency and without using radioactive labels. Homogeneity, as used herein, refers to the identical reactive groups (i.e., mercapto) located on one of the terminus of immunoglobulin G protein-binding molecules (e.g., peptides), which are covalently bonded to a substrate via the reactive groups. Efficiency, as used herein, refers to the ability of chimeric proteins to interact with the substrate or with the receptor binding ligand.

Other advantages, features, and objects of the invention will be apparent from the description and from the claims.

### DETAILED DESCRIPTION

This invention relates to a compound-bound substrate, and its use for preparing a receptor protein array and for identifying a receptor binding ligand.

Preparation of a Compound-Bound Substrate

The compound-bound substrate of this invention includes a solid support having a surface; and a plurality of compounds having the aforementioned formula (I) covalently bound to the surface. Referring to formula (I), an immobilized immunoglobulin G protein-binding molecule (referred to IgG-binding molecule hereinafter) includes a terminal sulfur atom connected to the surface via a maleimide group and a linking group. The distance, from the sulfur atom to the surface, is essentially determined by the length of the linking group. Suitable length of the linking group is selected such that immobilized the IgG-binding molecule can be used in combinatorial assays. Specifically, the optimal length may be determined by the efficiency of the Fc portion of an IgG protein binding to the IgG-binding molecule.

The linking group can be formed from any number or combination of atoms or molecules to provide an optimal distance between the substrate and the sulfur atom. For example, the linking group can be formed of organic polymers, e.g., repeating units of polyethylene glycol, —(OCH₂CH₂)ₙ—O—, to create acceptable hydrophilic conditions and appropriate length. In general, polyethylene glycol linking groups have between about 1 to about 12 repeating units.

The solid support can be a solid or porous solid support. In some implementations, the support is a bead, microparticle, a nanoparticle, a matrix, or a gel. Beads, microparticles, and nanoparticles can be used, e.g., in screening applications. Beads, matrices, and gels can be used, e.g., in purification methods, e.g., as a matrix for column chromatography. The beads can include interior surfaces that increase effective surface area and also partition components. The solid support used herein can be made from any material either flexible or rigid. In general, the material is resistant to the variety of synthesis and analysis conditions of assays. It, of course, can be a composite of one or more materials. For example, glass support, e.g., a glass slide, is coated with a polymer material to produce a solid support. Additionally, the solid support can be made in any shape, e.g., flat, tubular, round, and include etches, ridges or grids to create a patterned substrate. It can be opaque, translucent, or transparent. Further, the solid support can include wells or moats. See, e.g., Britland et al. (1992) Biotechno Prog 8: 155; Mooney et al. (1996) Proc Natl Acad Sci USA 93: 12287; Nicolau et al. (1998) Langmuir 14:1927; Williams et al. (1994) Biosens Bioelectron 9: 159; and Whaley et al. (2000) Nature 405: 665.

The compound-bound substrate described above can be prepared by a method disclosed herein. More specifically, the method includes the steps of: providing a solid support having a maleimide group; providing a plurality of IgG-binding molecules, each having a mercapto group at one of its termini (e.g., C-terminus); and contacting the IgG-binding molecules with the surface.

An IgG-binding molecule, including an IgG-binding domain of protein G, protein G, an IgG-binding domain of protein A, and protein A, can be a peptide that has a high affinity for an IgG protein. It can be prepared chemically (e.g., on a peptide synthesizer) or biologically (e.g., expressed from a host cell). An IgG-binding peptide can include a non-naturally occurring analog, e.g., a D-amino acid, an amino acid analog, or a peptidomimetic. A mercapto group at one of is termini can be introduced by, for example, incorporation of a mercapto-containing chemical group or a cysteine.

A solid support having a maleimide group can be prepared by a method delineated herein. One can react maleic anhydride with amino on the surface of the solid support (e.g., amino slides from Corning Inc. Life Science); followed by addition of a maleimide formation reagent (e.g., ZnBr₂/HMDS). The thus-prepared solid support can be used to immobilize polypeptides, oligonucleotides, or organic molecules, wherein each of the polypeptides, oligonucleotides, or organic molecules includes a mercapto group.

The methods described above may also additionally include steps, either before or after the steps described specifically herein, to add or remove suitable protecting groups in order to ultimately allow preparation of the compound-bound substrate of this invention. In addition, various steps may be performed in an alternate sequence or order. Synthetic chemistry transformations and protecting group methodologies (protection and deprotection) useful in preparing the aforementioned compound-bound substrate are known in the art and include, for example, those described in R. Larock, Comprehensive Organic Transformations, VCH Publishers (1989); T. W. Greene and P. G. M. Wuts, Protective Groups in Organic Synthesis, 2^{nd} Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Preparation of a Chimeric Protein

To practice the method of this invention, a chimeric protein, including a polypeptide containing the Fc portion of an IgG protein, and another polypeptide containing, e.g., a receptor protein (an extracellular domain of a receptor protein), can be prepared by a method known to a skilled person in the art. For example, the method includes exchanging the BamHI-XhoI restriction DNA fragments to obtain a recombinant nucleic acid encoding a mature chimeric protein described above. The recombinant nucleic acid is then ligated into a vector, e.g., pCEI expression vector. See, e.g., U.S. Pat. Nos. 5,580,756, 5,521,288, and 5,447,851.

A vector includes the recombinant nucleic acid in a form suitable for expression of the nucleic acid in a host cell. In particular, the vector may include one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the vector can depend on such factors as the choice of the host cell to be transformed, and the level of expression of protein desired. The vector can be introduced into a host cell to thereby produce the aforementioned chimeric protein.

The vector can be designed for expression of the chimeric protein in prokaryotic or eukaryotic host cells. For example, the chimeric protein is expressed in E. coli, insect cells (e.g., using baculovirus expression vectors), yeast cells, or mammalian cells (e.g., Chinese hamster ovary cells (CHO) or COS cells). Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

The term “host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A vector can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, and electroporation.

A host cell can be used to produce (i.e., express) the chimeric protein via the steps of culturing the host cell in a suitable medium such that the chimeric protein is produced and isolating the chimeric protein from the medium or the host cell.

The thus produced chimeric protein can then be purified by column chromatography (e.g., affinity column chromatography) or other techniques, if necessary. Purity can be readily measured by any appropriate method, for example, column chromatography, polyacryamide gel electrophoresis, or high-pressure liquid chromatography analysis.

A Screen Assay

The invention provides methods (also referred to herein as “screening assays”) for identifying ligands or test compounds (e.g., proteins, peptides, peptidomimetics, peptoids, small molecules or other drugs) which bind to a receptor protein, have a stimulatory or inhibitory effect on, for example, the binding between the receptor protein and its binding ligand, or have a stimulatory or inhibitory effect on, for example, the activity of the receptor protein. Ligands or compounds thus identified can be used in a therapeutic protocol or to elaborate the biological function of the receptor protein.

The ligands or test compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al. (1994) J. Med. Chem. 37:2678-85); spatially addressable parallel solid phase or solution phase libraries; the “one-bead one-compound” library method; and synthetic library methods using affinity chromatography selection. The biological library and peptoid library approaches are limited to peptide libraries, while the other approaches are applicable to peptide, non-peptide oligomer or small molecule libraries of compounds (Lam (1997) Anticancer Drug Des. 12:145). Examples of methods for the synthesis of molecular libraries can be found in the art, for example in: DeWitt et al. (1993) Proc. Natl. Acad. Sci. U.S.A. 90: 6909; Erb et al. (1994) Proc. Natl. Acad. Sci. USA 91: 11422; Zuckermann et al. (1994). J. Med. Chem. 37: 2678; Cho et al. (1993) Science 261: 1303; Carrell et al. (1994) Angew. Chem. Int. Ed. Engl. 33: 2059; Carell et al. (1994) Angew. Chem. Int. Ed. Engl. 33: 2061; and Gallop et al. (1994) J. Med. Chem. 37: 1233.

The ability of a ligand (or a test compound) to bind to a receptor protein with or without labeling of any of the interactants can be evaluated. For example, the ligand (or the test compound) is labeled. Rather than radioactive labels, labels such as fluorescence, chemiluminescence, or electrochemical luminescence can be used. Examples of fluorescent labels include fluoresceins, rhodamines (U.S. Pat. Nos. 5,366,860 and 5,936,087; 6,051,719), cyanines (U.S. Pat. No. 6,080,868 and WO 97/45539), and metal porphyrin complexes (WO 88/04777). In another example, the interaction between the ligand and the receptor protein is detected, e.g., using fluorescence energy transfer (FET) (see, U.S. Pat. Nos. 5,631,169 and 4,868,103), in which both the ligand and the receptor protein are labeled. A fluorophore label on the first “donor” molecule is selected such that its emitted fluorescent energy will be absorbed by a fluorophore label on a second “acceptor” molecule, which in turn is able to fluoresce due to the absorbed energy. Labels are chosen that emit different wavelengths of light, such that the “acceptor” molecule label may be differentiated from that of the “donor.” A FET binding event can be conveniently measured through standard fluorometric detection means well known in the art (e.g., using a fluorimeter). In a further example, determining the ability of the ligand to bind to the receptor protein can be accomplished using real-time Biomolecular Interaction Analysis (see, e.g., Sjolander & Urbaniczky (1991) Anal. Chem. 63: 2338-2345 and Szabo et al. (1995) Curr. Opin. Struct. Biol. 5: 699-705), in which neither the ligand nor the receptor protein is labeled. Changes in the mass at the binding surface (indicative of a binding event) result in alterations of the refractive index of light near the surface (the optical phenomenon of surface plasmon resonance), resulting in a detectable signal which can be used as an indication of real-time reactions between the ligand and the receptor protein.

An Array

Also within the scope of this invention is an array fabricated on a substrate of this invention. A chimeric protein can be deposited on the solid substrate in the form of an array. The array can be used in the screening assays described above. An array can have a density of at least 10, 50, 100, 200, 500, 10³, 10⁴, 10⁵, 10⁶, 10⁷, 10⁸, or 10⁹ addresses per cm², and/or a density of no more than 10, 50, 100, 200, 500, 10³, 10⁴, 10⁵, 10⁶, 10⁷, 10⁸, or 10⁹ addresses/cm². Preferably, the plurality of addresses includes at least 10, 100, 500, 1,000, 5,000, 10,000, or 50,000 addresses, or less than 9, 99, 499, 999, 4,999, 9,999, or 49,999 addresses. The center to center distance between addresses can be 5 cm, 1 cm, 100 mm, 10 mm, 1 mm, 10 nm, 1 nm, 0.1 nm or less and/or ranges between. The longest diameter of each address can be 5 cm, 1 cm, 100 mm, 10 mm, 1 mm, 10 nm, 1 nm, 0.1 nm or less and/or ranges between. Each address contains 10 mg, 1 mg, 100 ng, 1 ng, 100 pg, 10 pg, 0.1 pg, or less of a target compound and/or ranges between. Alternatively, each address contains 100, 10³, 10⁴, 10⁵, 10⁶, 10⁷, 10⁸, or 10⁹, or more molecules of the chimeric protein attached thereto and/or ranges between. Addresses in addition to addresses of the plurality can be deposited on the array. The addresses can be distributed, on the substrate in one dimension, e.g., a linear array; in two dimensions, e.g., a planar array; or in three dimensions, e.g., a three dimensional array.

A substrate with a planar surface described herein can be used to generate an array of a diverse set of receptor proteins or a limited set of receptor proteins. In one exemplary application, receptor proteins of differing sequence are positioned on the array surface. Such an array can be used to query one ligand or test compound. In anther example, receptor proteins of the same sequence are positioned on the array surface. Such an array can be used to query a plurality of ligands or test compounds.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

The invention will be further described in the following examples. It should be understood that these examples are for illustrative purposes only and are not to be construed as limiting this invention in any manner.

### EXAMPLES

Examples have been included to illustrate this invention. The example of chimeric IgG fusion receptor is the human TNF-receptor 1 extracellular domain (TNFR1-ED) and human IgG1 constant region (hinge, CH2 & CH3) fusion receptor (TNFR1-IgG). The example of derivatized glass slide is maleimide glass slide. The example of protein A is the cysteine-containing protein A. These examples are intended to be exemplary only and that shall lead us to the scope and spirit of this invention.

Preparation of a Chimeric IgG Fusion Receptor

The cDNA of TNFR1-ED was cloned from total RNA prepared from HL60 cell line using RT-PCR method. The primers used for the PCR reaction were:

5′end primer: 5′-GCGAGAGGATCCTGGCATGGGCCTCTCCACC-3′ (SEQ ID NO: 8)

3′end primer: 3′-GACTCCTGAGTCCGTGGTGGAGCTCTCTCGC-5′ (SEQ ID NO: 9)

A reverse transcription reaction was performed at 50° C. for 30 minutes and the reaction mixture was subjected to 30 cycles of polymerase chain reaction in a thermal cycler (Perkin Elmer) with a program: 50° C., 30 seconds; 68° C., 1 min, and 94° C., 30 seconds. The reaction mixture was analyzed in a gel electrophoresis, and then cleaved with restriction enzymes BamHI/XhoI and ligated into the expression vector pCEI (constructed by S. Y Shaw, unpublished). The pCEI plasmid encoding human IgG1 heavy chain constant region was prepared as previously described method (Seed & Arufo). The recombinant plasmid encoding TNFR1-IgG (pTNFR1-IgG) was linearlized with NheI cleavage and then transformed into CHO cells using electroporation method. The transformed cells resistant to 100 mM methotrexate was selected for production of TNFR1-IgG

Production and Purification of Chimeric IgG Fusion Receptors

The pTNFR1-IgG transformed cells were adapted into a serum free medium (Hyclone) over a two-week period. The adapted cells were grown in a 1 L spinner flask bioreactor to produce TNFR1-IgG protein. The conditioned medium from the reactor was passed through a protein A affinity column (Amersham Life Science). Bound protein was eluted with 0.1 M Glycine (pH 3.0) and then dialyzed against 10 mM phosphate buffer (pH 7).

Expression of Cysteine-Containing Protein A

The DNA encoding for protein A was cloned from total genomic DNA of Cowan I (ATCC12598) by a PCR method. The sequences of primers for the PCR reaction were:

| | |
|---|---|
| 3′ end primer: | (SEQ ID NO: 10) |

3′-CCATTTCTTCTGCCGTTGACAGGACCAATCCCTAGGTCTCGC-5′

| | |
|---|---|
| 5′ end primer: | (SEQ ID NO: 11) |

5′-GCGAGATCATGAAAAAGAAAAACATTTATTCAATTCG-3′

The 5′ end primer was corresponding to the 5′ end of protein A with added sequence to introduce a cysteine residue at the C-terminus of protein A. The PCR amplified DNA was digested with BspH1 and BamH1, and then ligated into BspHI/BamH1 site of pET 21 (Novagen) to obtain the expression plasmid pPASH. The pPASH was transformed into E. coli. BL21 for expression of cysteine-containing protein A.

Production and Purification of Cysteine-Containing Protein A (PA-SH)

The plasmid pPA-SH transformed E. coli. cells were grown in LB medium with ampicillin (0.5 mg/L) until optical density reached 0.3, and IPTG was added to induce the expression of PA-SH. After induction for 4 hrs, cells was harvested by centrifugation and then homogenized with a homogenizer (Microfluid System). The PA-SH in the supernatant was purified through an IgG affinity column (Amersham Life Science).

Preparation of Maleimide-Derivatized Glass Slides

Amine glass slide was derivatized as shown in Scheme 1 to give a surface that was fuctionalized with maleimide groups. In scheme 1, the maleimide-derivatized glass slide was prepared by reaction of maleic anhydride (1.6 mmole) with amine glass slide in toluene at room temperature. After 1 hour, ZnBr₂ (1.6 mmole) was added, and HDMS (2.4 mmole) was slowly added in 30 min to the reaction mixture. The reaction was refluxed at 100° C. for another hour. After the reaction, the slide was rinsed with water and dried under N₂.

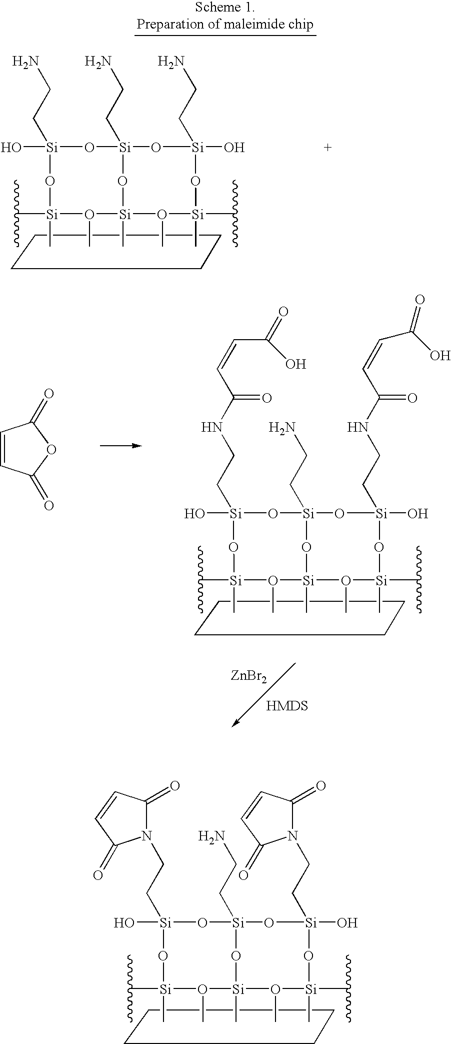

Immobilization of Cysteine-Containing Protein A (PA-SH) to Maleimide-Derivatized Glass Slide

Thiol-containing protein can readily attach to the maleimide-derivatized glass via the thioether linkage. The PA-SH was dissolved in 1 mL phosphate saline buffer (PBS) till final protein concentration of 1 mg/mL. Tris-(2-carboxethyl)phosphine (180 μg) was added to the PA-SH solution to reduce PA-SH protein. The reaction was performed at room temperature for 30 minutes, and it was then used to react with maleimide-derivatized glass slide at room temperature for one more hour. The protein A coated slide (PA slide) was rinsed with distilled water and blocked with 1% BSA in PBS.

Coating of TNFR1-IgG Fusion Receptor on the PA Slide

Coating of TNFR1-IgG to the PA slide is through the affinity interaction between IgG portion of TNFR1-IgG molecule and protein A molecules on the PA slide. The coating was performed by incubating the PA slide in TNFR1-IgG protein solution (1 mg/mL) at room temperature for 30 minutes. The TNFR1-coated slide was rinsed with PBS and distilled water, and stored under dry condition.

Printing of Fluorescence Labeled TNF-α to TNFR1-IgG Fusion Receptor Chip

TNF-α was labeled with a fluorescence dye Cy3 (Amersham Life Science). TNF-α (50 μg) was dissolved in 100 μL of sodium bicarbonate solution (20 mM, pH 6.8). Cy3 (1 mg) was added to the solution and incubated at room temperature. After one hour, 10 μL of Tris buffer (1 M, pH 8.0) was added to block the excess Cy3 at room temperature for 30 minutes. When the blocking reaction was completed, 1 mg of BSA was added and the reaction mixture was dialyzed against phosphate buffer (10 mM, pH 7.0). Cy3-labeled TNF-α at concentration range from 0.01 mg/mL to 0.5 mg/mL was used to examine its binding activity to TNFR1-IgG receptor chip. Microarray of Cy3-labeled TNF-α to the TNFR1-IgG receptor chip was performed at Affymetrix 417 (Affymetrix) with a 500 μm needle. The printed slides was washed in PBS plus 0.1% Tween 20, and then detected in a fluorescence scanner (Axon). The result shows that the binding Cy3-labeled TNF-α to TNFR1-IgG receptor chip reaches its maximum of binding at concentration of 0.1 mg/mL.

Competitive Binding of Fluorescence Labeled TNF-α to TNFR1-IgG Receptor Chip

The competitive binding of fluorescence labeled TNF-α to TNFR1-IgG fusion receptor was performed by mixing Cy3-labeled TNF-α (0.05 mg/mL) with equal volume of various concentration of unlabeled TNF-α (0.01 to 0.5 mg/mL). The mixtures were printed on a TNFR1-IgG receptor chip by a microarrayer (Affymetrix) with a 500 μm needle. The printed slides was washed in PBS plus 0.1% Tween 20, dried in the air and then detected in a fluorescence scanner (Axon). The result shows that the binding of Cy3-labeled TNF-α to TNFR1-IgG receptor can be blocked by unlabeled TNF-α in a dose response manner.

Other Embodiments

All of the features disclosed in this specification may be used in any combination. Each feature disclosed in this specification may be replace by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Accordingly, other embodiments are also within the scope of the following claims.

What is claimed is:

1. A compound-bound substrate comprising:
a solid support having a surface; and
a plurality of compounds having formula (I) covalently bound to the surface: 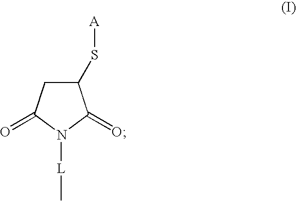
wherein
L is a linking group or a bond; and
A is an immunoglobulin G protein-binding molecule that is covalently bonded at one of its termini.

2. The compound-bound substrate of claim 1, further comprising a chimeric protein that includes a first polypeptide containing the Fc portion of an immunoglobulin G protein, wherein the chimeric protein binds to the surface.

3. The compound-bound substrate of claim 2, wherein the chimeric protein further comprises a second polypeptide having a receptor protein.

4. The compound-bound substrate of claim 3, wherein the second polypeptide is an extracellular domain of a receptor protein.

5. The compound-bound substrate of claim 4, wherein the receptor protein is a type I membrane protein.

6. The compound-bound substrate of claim 5, wherein the receptor protein is tumor necrosis factor-alpha.

7. The compound-bound substrate of claim 5, wherein the immunoglobulin G protein-binding molecule is protein A or protein G.

8. The compound-bound substrate of claim 5, wherein the immunoglobulin G protein-binding molecule is a peptide comprising the amino acid sequence of SEQ ID NO: 1, SEQ ID NO: 2, SEQ ID NO: 3, SEQ ID NO: 4, SEQ ID NO: 5, SEQ ID NO: 6, or SEQ ID NO: 7.

9. The compound-bound substrate of claim 1, wherein the immunoglobulin G protein-binding molecule is a peptide that is covalently bonded to the sulfur atom at its C-terminus.

10. The compound-bound substrate of claim 9, further comprising a chimeric protein that includes a first polypeptide containing the Fc portion of an immunoglobulin G protein, wherein the chimeric protein binds to the surface.

11. The compound-bound substrate of claim 10, wherein the chimeric protein further comprises a second polypeptide having a receptor protein.

12. The compound-bound substrate of claim 11, wherein the second polypeptide is an extracellular domain of a receptor protein.

13. The compound-bound substrate of claim 12, wherein the receptor protein is a type I membrane protein.

14. The compound-bound substrate of claim 13, wherein the receptor protein is tumor necrosis factor-alpha.

15. The compound-bound substrate of claim 13, wherein the immunoglobulin G protein-binding molecule is protein A or protein G.

16. The compound-bound substrate of claim 13, wherein the immunoglobulin G protein-binding molecule is a peptide comprising the amino acid sequence of SEQ ID NO: 1, SEQ ID NO: 2, SEQ ID NO: 3, SEQ ID NO: 4, SEQ ID NO: 5, SEQ ID NO: 6, or SEQ ID NO: 7.

17. A compound-bound substrate made by a process comprising the steps of:
providing a solid support having a surface that comprises a chemical group of formula -L-X; wherein L is a linker group or a bond and X is a maleimide group;
providing a plurality of immunoglobulin G protein-binding molecules, each having a mercapto group at one of its termini; and
contacting the immunoglobulin G protein-binding molecules with the surface.

18. The compound-bound substrate of claim 17, further comprising the steps of:
providing a chimeric protein that includes a first polypeptide having the Fc portion of an immunoglobulin G protein and a second polypeptide having a receptor protein; and
contacting the chimeric protein with the surface.

19. A method for preparing a substrate comprising the steps of:
(a) providing a surface having a plurality of molecules that include a chemical group of formula -L-NH₂, wherein L is a linking group or a bond;
(b) contacting maleic anhydride with the surface; and
(c) contacting a maleimide formation reagent with the surface.

20. The method of claim 19, further comprising contacting a plurality of polypeptides with the surface after step (c), wherein each of the polypeptides includes a mercapto group.

21. The method of claim 20, wherein each polypeptide includes at most one mercapto group.

22. The method of claim 20, wherein each polypeptide includes the mercapto group at one of its termini.

23. The method of claim 22, wherein each polypeptide includes the mercapto group at its C-terminus only.

24. A substrate comprising:
a compound-bound substrate of claim 1; and
a chimeric protein that includes a first polypeptide having the Fc portion of an immunoglobulin G protein and a second polypeptide having a receptor protein, in which the chimeric protein binds to the compound-bound substrate;
wherein the bound chimeric proteins have a density of at least 5×10¹⁵ molecules/cm².

25. The substrate of claim 24, wherein the immunoglobulin G protein-binding molecule is a peptide that is covalently bonded to the sulfur atom at its C-terminus.

26. The substrate of claim 24, wherein the second polypeptide is an extracellular domain of a receptor protein.

27. The substrate of claim 26, wherein the receptor protein is a type I membrane protein.

28. The substrate of claim 27, wherein the receptor protein is tumor necrosis factor-alpha.

29. The substrate of claim 27, wherein the immunoglobulin G protein-binding molecule is protein A or protein G.

30. The substrate of claim 27, wherein the immunoglobulin G protein-binding molecule is a peptide comprising the amino acid sequence of SEQ ID NO: 1, SEQ ID NO: 2, SEQ ID NO: 3, SEQ ID NO: 4, SEQ ID NO: 5., SEQ ID NO: 6 or SEQ ID NO: 7.

31. An array comprising:
a substrate having a plurality of addressable sites;
each addressable site having a compound of formula (I): 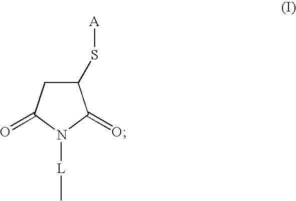
in which L is a linking group or a bond; and A is an immunoglobulin G protein-binding molecule that is covalently bonded at one of its termini; and
each addressable site having a chimeric protein that includes a first polypeptide having the Fe portion of an immunoglobulin G protein, and a second polypeptide having a receptor protein, in which the chimeric protein binds to the immunoglobulin G protein-binding molecule.

32. The array of claim 31, wherein the receptor protein is unique among each addressable site.

33. The array of claim 31, wherein the receptor protein is identical among each addressable site.

34. The array of claim 31, wherein the immunoglobulin G protein-binding molecule is a peptide that is covalently bonded to the sulfur atom at its C-terminus.

35. The array of claim 34, wherein the bound chimeric proteins have a density of at least 5×10¹⁵ molecules/cm².

36. A method for identifying a receptor binding ligand, comprising:
contacting a ligand with a compound-bound substrate of claim 3; and
determining whether the ligand binds to the receptor protein.

37. The method of claim 36, wherein the ligand is fluorescence-labeled.

38. A method for identifying a compound that inhibits the binding of a receptor binding ligand to a receptor protein, comprising:
contacting a test compound and a receptor binding ligand with a compound-bound substrate of claim 3; and
determining whether the ligand binding is different from that without the presence of the test compound.

39. The method of claim 38, wherein the ligand is fluorescence labeled.

40. The method of claim 38, wherein the test compound is fluorescence labeled.

41. The method of claim 38, wherein the test compound is a polypeptide.

42. The method of claim 38, wherein the test compound is a small organic molecule.

43. A kit for testing the ability of a compound to bind to a receptor, comprising a compound-bound substrate of claim 1.
